(19) United States
(12) Patent Application Publication
(10) Pub. No.: US 2026/0207186 A1
(43) Pub. Date: Jul. 23, 2026

(54) MULTI-VECTOR DYNAMIC TRACTION DEVICES, SYSTEMS, AND METHODS

(71) Applicant: BOSTON SCIENTIFIC MEDICAL DEVICE LIMITED, Galway (IE)

(72) Inventors: Deepak Kumar Sharma, Muzaffarnagar (IN); Sharath Kumar G, Kankapura (IN)

(73) Assignee: BOSTON SCIENTIFIC MEDICAL DEVICE LIMITED, Galway (IE)

(21) Appl. No.: 19/453,213

(22) Filed: Jan. 20, 2026

Related U.S. Application Data
(60) Provisional application No. 63/747,638, filed on Jan. 21, 2025.

(51) Int. Cl. A61B 17/02; A61B 1/00; A61B 17/00

(52) U.S. Cl. CPC A61B 17/0293; A61B 1/0014; A61B 17/00234; A61B 17/0218; A61B 2017/00269; A61B 2017/00296

(57) ABSTRACT

A traction device and system configured for use with a medical delivery device having a lumen through which medical instruments are delivered. The traction device and system allow for delivery and/or deployment of the traction device independently of delivery of medical instruments through the medical delivery device. Additionally or alternatively, the traction device and system allow for manipulation and/or control of the traction device independently of manipulation and control of other medical instruments. Additionally or alternatively, the traction device and system allow for application of multi-vector traction to a target tissue. The traction device may be controlled from outside the patient’s body to apply traction to target tissue within a patient’s body.

### CROSS-REFERENCE TO RELATED APPLICATIONS

This application claims the benefit of priority under 35 U.S.C. § 119 to U.S. Provisional Application No. 63/747,638, filed on January 21, 2025, the entire disclosure of which is hereby incorporated by reference herein for all purposes.

### FIELD

The present disclosure relates generally to the field of medical devices, systems, and methods for applying traction to tissue.

### BACKGROUND

Various endoscopic surgical procedures require maneuvering of medical instruments about various anatomical structures within a limited space within a patient’s body. In procedures involving cutting of tissue (e.g., endoscopic mucosal resection (EMR), Endoscopic Submucosal Dissection (ESD), Pre-Oral Endoscopic Myotomy (POEM), etc.), one of the largest time and complexity drivers is managing the tissue being cut. The loose section of tissue may obstruct visibility, such as by falling on the endoscope, occluding visibility of the camera and creating a hindrance affecting movement of the instruments used during the procedure and in reaching all regions and depths of the target tissue being cut. Positioning of a traction device to lift the cut (and often hanging) mass of tissue, thus clearing the path for visibility and operation of medical tools and devices, may be challenging, particularly in a space-restricted environment. During endoluminal surgery procedures, typically, the physician holds and manipulates the scope with the left hand and manipulates the shaft (insertion tube) of the scope with the right hand to get the scope in the target position. Once the scope is in the desired location, a technician hands over the relevant accessories (snare, radiofrequency (RF) knife, etc.) to the physician who then introduces the accessory into the scope and positions the accessory, such as by torquing the shaft, accessory, or scope handle, for use in the procedure. Presently, getting the accessory in the right position is a combination of scope handle manipulation, shaft manipulation, accessory manipulation, and accessory torquing. To complete the procedure/task, the accessory needs to be actuated. A medical technician generally investigates preparing the accessory before introducing it into the scope, actuates the accessory, and takes care of injecting saline or specific medium into the accessory based on the physician’s instruction and the procedure, requiring coordination of both medical professionals, and thus further complicating the procedure. Some procedures may be limited to being performed with a medical scope which has a single working channel. In order to be able to maximize use of the working channel for tools for performing the procedure, an over-the-scope traction system may be used. A resecting tool may be delivered through the scope, and the traction device over the scope. For instance, a filament is attached to a tissue clip, and the filament and clip are mounted along the exterior of the medical scope for delivery to the treatment site, leaving the working channel available for delivery of medical instruments such as tissue resecting tools. The clip is attached to tissue, and the filament, which is attached to the tissue via the clip, extends proximally to the medical professional for access outside the patient. The medical professional may exert proximal force on the filament to increase tension on the tissue. Since the traction device is attached to the endoscope, it is difficult to maintain continuous traction during endoscope movement. Also, although the medical professional may vary the magnitude of the force applied to the filament, and thus to the tissue, the direction of the force is generally limited to essentially a single direction. To apply a force in more than one direction, the medical scope must be removed so that another filament and clip may be mounted on the medical scope, and the medical scope must then be reinserted into the patient to deliver the clip and filament, and to attach the clip to another location along the treatment site so that the filament may be pulled to apply traction to the tissue at the other location. Solutions to these and other challenges in the art would be welcome.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a traction system is disclosed for delivery into a patient’s body with a medical delivery device. The medical delivery device may have a lumen for delivering medical instruments through a distal end of the medical delivery device. In some aspects, the traction system includes a distal interface member comprising a wall configured to interface with the medical delivery device; a first anchor element mounted on an exterior surface of the distal interface member; and a first elongate element operably associated with the first anchor element and extendable proximally from the distal interface member for access outside the patient’s body.

In some aspects, the first anchor element extends circumferentially around the exterior surface of the distal interface member. In some aspects, the first anchor element is in the form of a band or loop encircling an opening. In some aspects, the first anchor element is structurally self-supporting to maintain the opening in an open configuration without the first anchor folding on itself.

In some aspects, the first anchor element is structurally self-supporting to maintain a selected shape without folding on itself.

In some aspects, the distal interface member is configured to extend circumferentially around the exterior of a portion of the medical delivery device. in some aspects, the wall of the distal interface member is tubular and shaped to extend circumferentially around the distal end of the medical delivery device.

In some aspects, a first radially-outwardly extending stopper is defined on the exterior surface of the distal interface member to retain the first anchor element in place with respect to the distal interface member. In some aspects, the first radially-outwardly extending stopper is an elevated ridge extending circumferentially around the exterior of the distal interface member.

In some aspects, the first elongate element extends distally from the first anchor element, and around a direction-changing feature defined with respect to the distal interface member to change directions to extend proximally to outside the patient’s body. In some aspects, the direction-changing feature is defined between a pair of slits in a distal end of the distal interface member.

In some aspects, the traction system further includes a second anchor element mounted on the exterior surface of the distal interface member, and a second elongate element operably associated with the second anchor element and extendable proximally from the anchor element on the distal interface member for access outside the patient’s body.

In accordance with various principles of the present disclosure, a traction system for delivery into a patient’s body includes a proximal interface member configured to interface with a medical delivery device; a distal interface member comprising a wall configured to interface with the medical delivery device; a first anchor element mounted on an exterior surface of the distal interface member; and a first elongate element operably associated with the first anchor element and extendable proximally from the distal interface member to the proximal interface member.

In some aspects, the first anchor element and the first elongate element are preassembled on the distal interface member such that the traction system is assembled and ready for operable association with a medical delivery device to operably associate the first anchor element and the first elongate element with the medical delivery device.

In some aspects, the traction system further includes a medical delivery device having a lumen for delivering medical instruments through a distal end of the medical delivery device, wherein the first elongate element extends along the exterior of the medical delivery device outside the lumen of the medical delivery device.

In some aspects, the traction system further includes a second anchor element mounted on the exterior surface of the distal interface member, and a second elongate element operably associated with the second anchor element and extendable proximally from the anchor element on the distal interface member for access outside the patient’s body.

In accordance with various principles of the present disclosure, a method is disclosed for assembling a traction device with respect to a medical delivery device having a lumen for delivering medical instruments through a distal end of the medical delivery device. In some aspects, the method includes mounting a distal interface member with respect to the distal end of

the medical delivery device, the distal interface member having a first anchor element of a traction device extending circumferentially around the exterior of the distal interface member; and extending a first elongate element from a distal end operably coupled with the first anchor element to the proximal end of the medical delivery device.

In some aspects, the distal interface member includes a direction-changing feature, the first elongate element extending distally from the first anchor element to around the direction-changing feature, and then proximally.

In some aspects, the distal interface member includes a second anchor element positioned proximal to the first anchor element, with a second elongate element operably associated with the second anchor element and extending proximally.

In some aspects, the method further includes mounting a proximal interface member with respect to a proximal end of the medical delivery device and extending the first elongate element to the proximal interface member for control of the elongate element along the proximal interface member.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within and for delivery into a patient’s body. For purposes of clarity and simplicity, not every element is labeled in every figure, nor is every element of each embodiment shown where illustration is not necessary to allow those of ordinary skill in the art to understand the disclosure. Moreover, certain features in one Figure may be used across different Figures and are not necessarily individually labeled when appearing in different Figures. For the sake of brevity and convenience, and without intent to limit, the descriptions of common features across the Figures are generally not repeated.

The detailed description will be better understood in conjunction with the accompanying drawings, wherein like reference characters represent like elements, as follows:

FIG. 1 illustrates a perspective view of an example of an embodiment of a traction device and system formed in accordance with aspects of the present disclosure.

FIGS. 2A-2D illustrate a schematic representation of a treatment site and one or more traction devices deployed with respect to the treatment site.

FIG. 3 is a schematic illustration of a view of a treatment site from the perspective of a medical delivery device along which the illustrated examples of embodiments of traction devices may be delivered.

FIG. 4 illustrates a perspective view of a distal interface member of a traction system with two traction devices operably associated therewith for delivery to a treatment site.

FIG. 5 is a view similar to FIG. 4, but with the distal traction device deployed.

FIG. 6 is a view similar to FIG. 5, but with the proximal traction device deployed as well.

FIG. 7 is a perspective view of a proximal interface member of an example of an embodiment of a traction system formed in accordance with various principles of the present disclosure and mounted with respect to a medical delivery device.

FIG. 8 is a side view of the proximal interface member and medical delivery device illustrated in FIG. 7.

FIG. 9 is another perspective view of the proximal interface member and medical delivery device illustrated in FIG. 7 and FIG. 8.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, proximate, etc.) such location or site. As understood herein, corresponding is intended to convey a relationship between components, parts, elements, etc., configured to interact with or to have another intended relationship with one another.

The present disclosure describes devices, systems, and methods for applying traction to tissue, such as within a patient’s body. In some aspects, the devices, systems, and methods are configured to apply multi-vector traction to tissue. In some aspects, the devices, systems, and methods are configured for delivery of a traction device over and along the exterior of a medical delivery device, leaving a lumen through the medical delivery device available for passage of medical instruments therethrough without the traction device extending through such lumen. The medical delivery device may be any appropriate flexible tubular elongate member capable of being navigated within a patient’s body (e.g., through curved and/or tortuous passageways within the patient’s body) and defining a lumen therethrough for delivery of one or more medical instruments. For the sake of convenience, and without intent to limit, reference is made to a medical scope as the medical delivery device. Delivery of a medical device, such as the traction device of the present disclosure, along the exterior of a medical scope is typically known in the art as “over-the-scope” delivery. Reference to a medical scope is as a generic term for a device with a flexible tubular elongate member or shaft insertable into a patient’s body, and optionally with additional features (e.g., working channels, suction / irrigation channels, an illuminating device, a visualization device, etc., the present disclosure not being limited in this regard). Various examples of medical scopes include, without limitation, endoscopes, arthroscopes, bronchoscopes, colonoscopes, cystoscopes, duodenoscopes, gastroscopes, hysteroscopes, laparoscopes, ureteroscopes, etc. It will be appreciated that terms such as medical tools, instruments, devices, accessories, etc., may be usable interchangeably herein, without intent to limit.

In some aspects, more than one traction device is delivered at the same time. More particularly, in some aspects, more than one traction device may be mounted with respect to the exterior of a medical scope for delivery to a treatment site so that more than one traction device is available for delivery once the distal end of the medical scope has been delivered to the treatment site, without the need to remove the distal end of the medical scope.

In some aspects, a traction device and system formed in accordance with various principles of the present disclosure includes a proximal interface and a distal interface for operably associating with a medical delivery device such as a medical scope. The traction device extends from the distal interface to the proximal interface. In some aspects, the traction device includes a tissue-engaging anchor element and an elongate element extending proximally from the anchor element to the proximal interface. The elongate element may be any elongated flexible element capable of transmitting a force to tissue to which the filament is operably coupled, and may be any of a filament, wire, cord, cable, elastic band, stretchable band, suture, music wire, muscle wire, dental floss, etc., the present disclosure not being limited in this regard. The distal interface may be in the form of a cap, such as an endcap for a medical scope. The proximal interface may be in the form of a control handle or other interface which may be operably associated with a control handle of the medical delivery device, and configured to be operably associated with the traction device for operation / manipulation of the traction device (e.g., application of traction force thereto). For instance, the proximal end of the elongate element may be operably associated with the proximal interface to allow the medical professional to apply proximal force to the elongate element to apply traction to tissue once the tissue-engaging element is coupled with tissue. The system, including the proximal and distal interfaces and the traction device, may be supplied, such as in the form of a kit, or preassembled and ready for operable association with the medical delivery device for use during a medical procedure, or preassembled with the medical delivery device. As such, the medical professional need not fit an anchor element or an elongate element into a working channel of a medical scope, and may simply mount the system for ready use for deploying a traction device, and applying traction to tissue via the traction device during a procedure.

Various embodiments of traction devices, systems, and methods will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

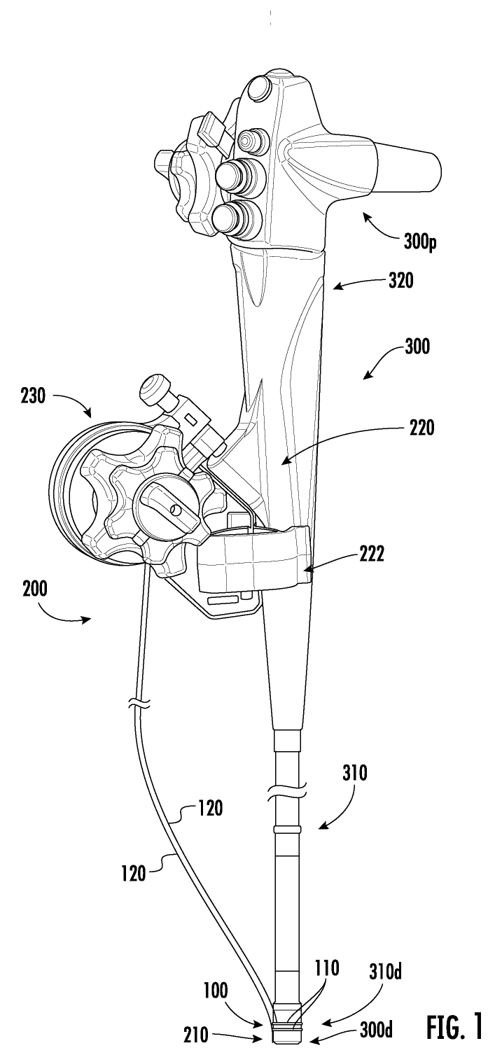

Turning now to the drawings, an example of an embodiment of a traction device 100 formed in accordance with various principles of the present disclosure is illustrated in FIG. 1. The traction device 100 is illustrated as a part of, or at least operably associated with (mounted with respect to and/or for delivery with and/or for delivery by), an example of an embodiment of a traction system 200 formed in accordance with various principles of the present disclosure. The traction device 100 and the traction system 200 may be delivered to a treatment site in conjunction with a medical delivery device 300. The example of an embodiment of a medical delivery device 300 illustrated in FIG. 1 is a medical scope, with a distal end 300d having a flexible tubular elongate member 310 configured for insertion into, and navigating within tortuous or at least curved portions of, a patient’s body (also known as an insertion tube). The proximal end 300p of the medical delivery device 300 may include a control handle 320 which may be used to navigate the flexible tubular elongate member 310 within the patient’s body and/or to operate one or more operable features (e.g., suction, irrigation, light, visualization device, etc.) of the medical delivery device 300. However, it will be appreciated that the present disclosure is not limited in this regard.

In accordance with various principles of the present disclosure, the traction device 100 is configured to be coupled to tissue at a treatment site for application of traction force to the treatment site. The traction device 100 includes an anchor element 110 and an elongate element 120 operably coupled with the anchor element 110. The elongate element 120 may be looped around a portion of the anchor element 110 and knotted, adhered, welded, etc., to be secured with respect to the anchor element 110; and/or extended through an aperture in a portion of the anchor element 110 and knotted (or otherwise modified to increase the thickness thereof, such as fused, crimped, etc.); and/or otherwise operably coupled (e.g., fixed) with respect to the anchor element 110. In some aspects, the anchor element 110 is configured to be engaged by a tissue-engaging device, so that the tissue-engaging device may engage tissue and thereby anchor the anchor element 110 with respect to tissue at the treatment site. It will be appreciated that terms such as engage (and other grammatical forms thereof) may be used interchangeably herein with terms such as, without limitation, couple, grasp, hold, clasp, clip, anchor, attach, affix, secure, etc. (and other grammatical forms thereof), without intent to limit. The elongate element 120 extends proximally from the anchor element 110 to outside the patient’s body so that the medical professional may apply a force to the anchor element 110 of the traction device 100 via the elongate element 120 to apply traction to tissue to which the anchor element 110 is anchored.

In accordance with various principles of the present disclosure, the traction device 100 is deliverable to a treatment site within a patient independently of (and not affecting or being affected by) delivery of other instruments to the treatment site. In some aspects, medical instruments with end effectors for performing a procedure with respect to tissue at the treatment site may be delivered through a lumen of the medical delivery device 300 (e.g., the working channel of a medical scope), whereas the traction device 100 of the present disclosure is not delivered through the same lumen through which such medical instruments are delivered, but, instead, is delivered along another path, channel, mechanism, etc. For instance, in some aspects, a traction device 100 of the present disclosure may be configured for delivery outside the lumen of a medical delivery device 300 through which other medical instruments are delivered. In some aspects, the traction device 100 is configured for delivery external to (i.e., along the exterior surface of) the medical delivery device 300. In some aspects, the traction device 100 is delivered through a lumen of a multi-lumen medical delivery device 300 which is separate and independent of a lumen through which other medical instruments, such as with end effectors for performing a procedure (e.g., cutting) tissue, are delivered. As such, the delivery of the traction device 100 may be independent of delivery of other medical instruments so that the medical professional may deliver and use medical instruments without interfering with and/or without interference of the traction device 100. Extension of the elongate element 120 of the traction device 100 along a path separate, apart, and distinct from (not coextensive with) the path of instruments for performing the procedure enhances the medical professional’s ability to adjust tension independently of the ability to operate other medical instruments in performing a procedure.

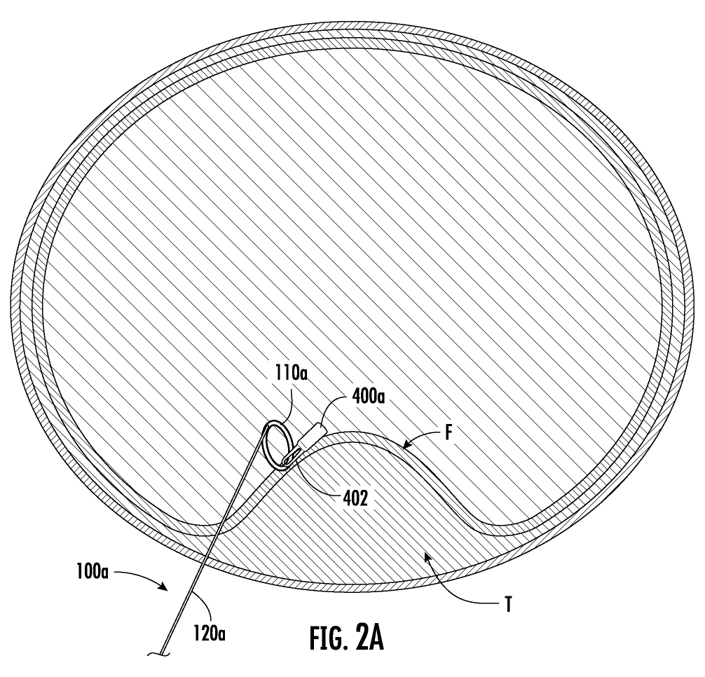

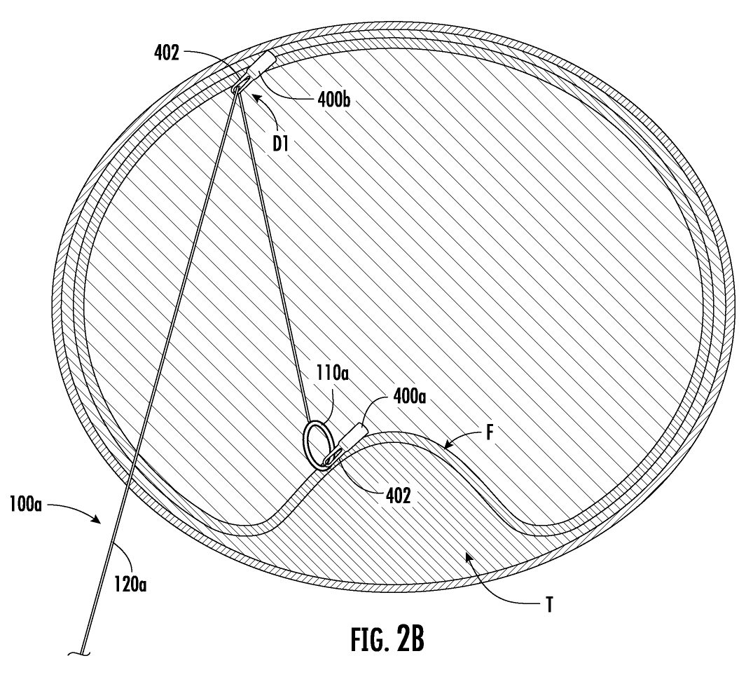

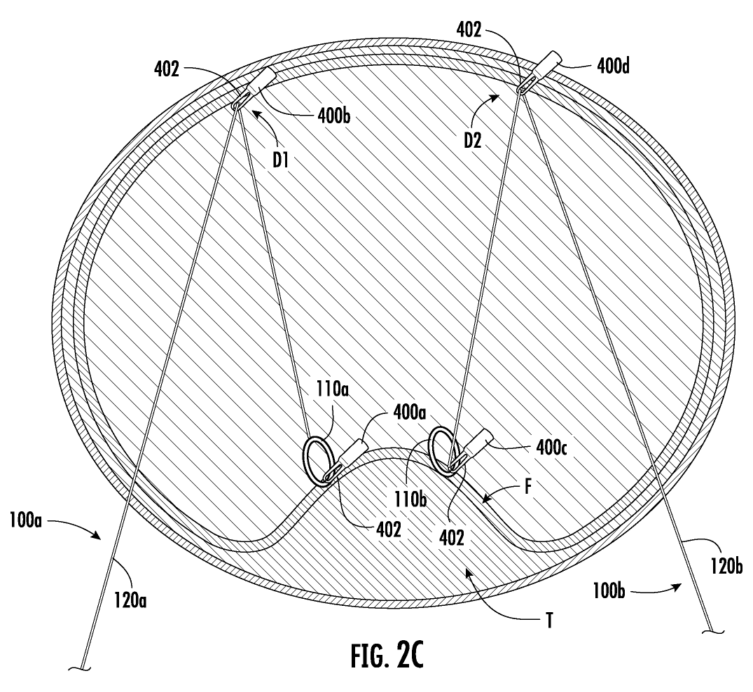

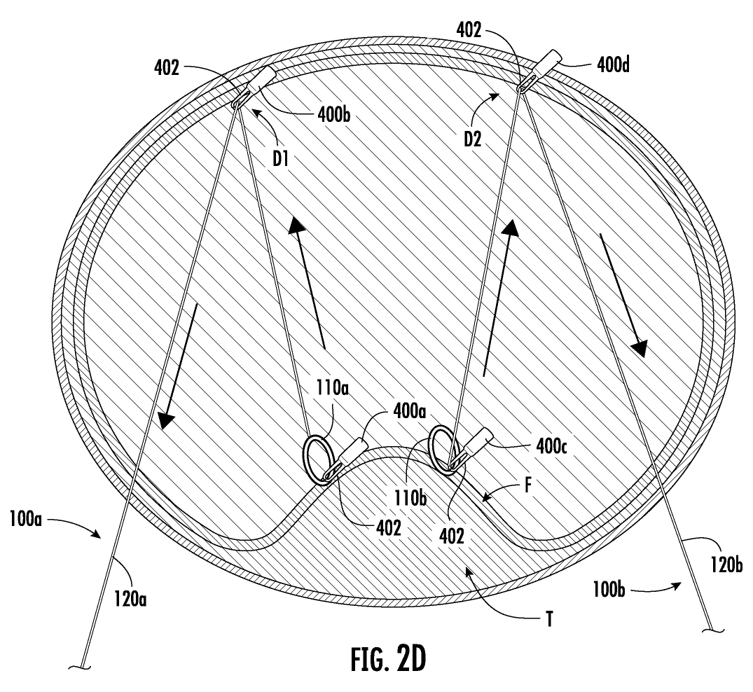

In use, the traction device 100 is anchored with respect to target tissue F at a treatment site T at which a medical procedure is being performed, such as schematically illustrated in FIGS. 2A-2D. In FIGS. 2A-2D, the treatment site T is schematically illustrated as a body lumen at which a flap of tissue, the target tissue F, is being cut away from underlying tissue, such as may occur during an endoluminal submucosal dissection (ESD) procedure. Traction is applied to the target tissue F to lift the tissue flap F away from underlying tissue with respect to which a procedure is to be performed (e.g., further cutting, such as to remove the target tissue F). In some aspects, the anchor element 110 of the traction device 100 is anchored with respect to the target tissue F, and traction is applied to the anchor element 110a, and thus the target tissue F, via the elongate element 120a. The traction device 100 (e.g., the anchor element 110 of the traction device 100) may be anchored with respect to target tissue F with the aid of a tissue-engaging device 400, such as illustrated in FIG. 2A. The example of an embodiment of a tissue-engaging device 400 illustrated in FIG. 2A is a tissue clip with jaws 402 which are configured to grasp both the anchor element 110 of the traction device 100 as well as tissue. However, other forms and/or configurations of tissue-engaging devices known to those of ordinary skill in the art may be used, the present disclosure not being limited in this regard. It will become apparent that the traction device 100 and tissue-engaging device 400 are indicated in FIG. 2A with the letter “a” as a suffix because additional traction devices 100 and additional tissue-engaging devices 400 may be deployed at the treatment site T to apply multi-vector traction to the target tissue F. Reference is thus made to traction device 100a (and its elements 110a, 120a) and tissue-engaging device 400a with reference to FIG. 2A, with the understanding that the letter “a” is only to differentiate the traction device 100 (and its elements) and the tissue-engaging device 400 illustrated in FIG. 2A from subsequently deployed traction devices 100 and tissue-engaging devices 400.

In accordance with various principles of the present disclosure, while the anchor element 110a is anchored to target tissue F, such as illustrated in FIG. 2A, the elongate element 120a extends proximally to outside the patient for access by a medical professional. The medical professional may apply a traction force to a proximal portion of the elongate element 120a accessible to the medical professional, and thereby may apply traction to the anchor element 110a and the target tissue F. If the medical professional needs to alter the direction of the force applied to the target tissue F by the anchor element 110a, a second tissue- engaging device 400b may be delivered to the treatment site T to grasp a portion of the elongate element 120a and then engage tissue at a first direction-changing location D1 spaced apart from the target tissue F, such as illustrated in FIG. 2B. In some aspects, the second tissue-engaging device 400b grasps the elongate element 120a in a manner which allows movement of the elongate element 120b with respect to the jaws 402 of the second tissue-engaging device 400b. Continued movement of the elongate element 120a with respect to the second tissue-engaging device 400b allows movement of the portion of the elongate element 120a extending from the second tissue-engaging device 400b to the anchor element 110a and the first tissue-engaging device 400a to adjust the magnitude of the force applied to the anchor element 110a and the target tissue F. As may be appreciated, such as with reference to FIG. 2B, because the second tissue-engaging device 400b holds a portion of the elongate element 120a at a second location with respect to the target tissue T, the direction of the force initially applied to the target tissue F is altered.

Further tissue-engaging devices 400 may be used to further alter the direction of force applied by the elongate element 120a to the anchor element 110a and the target tissue F. Additionally or alternatively, a second traction device 100b may be delivered and deployed at the treatment site T, such as illustrated in FIG. 2C. The second traction device 100b is illustrated already deployed, with a third tissue-engaging device 400c anchoring the anchor element 110b with respect to the target tissue F, and with a fourth tissue-engaging device 400d holding a portion of the elongate element 120b extending away from the anchor element 110b at a second direction-changing location D2. However, it will be appreciated that traction may be applied to the second traction device 100b without the use of a fourth tissue-engaging device 400d. The above descriptions of the first traction device 100a are applicable mutatis mutandis to the second traction device 100b, reference being made thereto for the sake of brevity, and without intent to limit.

As illustrated schematically in FIG. 2D, with two traction devices 100 deployed at the treatment site T and engaging the target tissue F, a medical professional may apply multi-vector traction to the target tissue F, such as represented by the directional arrows along the elongate elements 120. As may be appreciated, the elongate elements 120a, 120b are independent and separate from (external to, not coupled to, not operatively associated with, etc.) a medical delivery device.

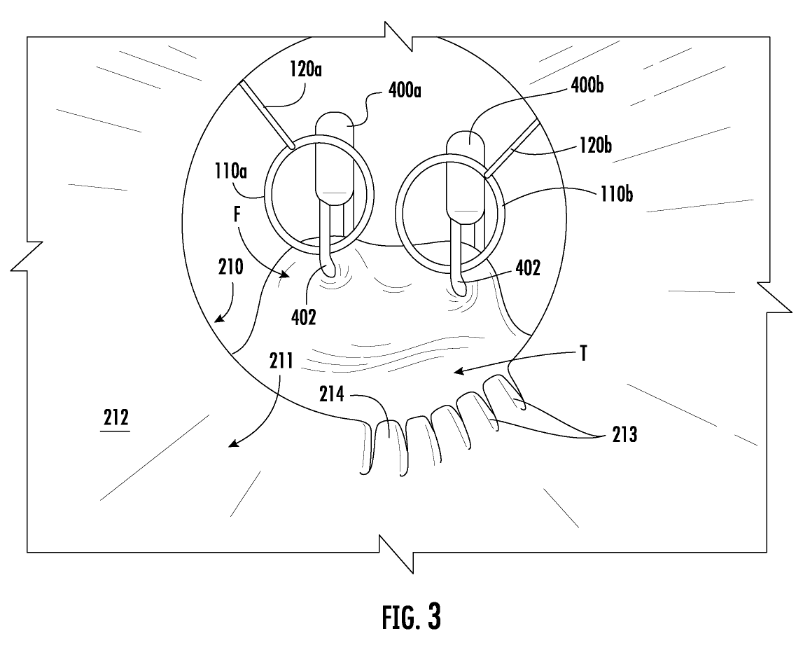

A medical professional may apply force to (or release force on) the elongate element 120 to apply multi-vector, dynamic traction on the target tissue F while maintaining visualization of the cutting plane and separating the target tissue F from the treatment site T, such as illustrated in FIG. 3, showing a schematic view from the distal end of a medical delivery device 300 (such as illustrated in FIG. 1) with which the traction device 100 is used. The medical professional thereby can remove obstructions which would otherwise be created by the flap of target tissue F to allow access to the treatment site T by a medical instrument delivered through a lumen (e.g., working channel) defined through the medical delivery device 300. As may be appreciated, the elongate element 120 extends along a path which is not through the lumen through which medical instruments are delivered for performing a procedure at the treatment site T.

As noted above, the traction device 100 of the present disclosure may be delivered in conjunction with a medical delivery device 300, such as illustrated in FIG. 1. In accordance with various principles of the present disclosure, the traction device 100 is delivered independently of delivery of other medical instruments. For instance, the traction device 100 is not delivered to a treatment site through the same lumen through which the medical professional delivers other medical instruments (e.g., with end effectors for performing a procedure with respect to the treatment site, such as a cutting instrument). In some aspects, the traction device 100 is delivered outside the lumen through which other medical instruments are delivered. For instance, the traction device 100 may be delivered external to the medical delivery device 300, such as illustrated in FIG. 1. In some aspects, the traction device 100 is delivered by a traction system 200 which delivers the traction device 100 with (e.g., by being coupled to) the flexible tubular elongate member 310 of the medical delivery device 300 (which is inserted into the patient’s body). In some aspects, the traction system 200 operates to deliver, deploy, manipulate, control, etc., the traction device 100 independently of operation, movement, manipulation, control, etc., of the medical delivery device 300 for delivery of other instruments. In some aspects, the traction device 100 and/or the traction system 200 is configured so that two or more traction devices 100 are deliverable and/or operable without interruption of use of the medical delivery device 300 (e.g., without the need to remove or reposition the medical delivery device 300). In some aspects, the traction device 100 and/or the traction system 200 is configured so that the traction device 100 is deliverable along the exterior of the medical delivery device 300.

Additional tissue-engaging devices 400 of a traction system 200 of the present disclosure, for altering traction force vectors, may be delivered external to / along the exterior of the medical delivery device 300 and/or through a working channel of the medical delivery device 300. In embodiments of a traction system 200 delivered with a multi-lumen medical delivery device 300, the additional tissue-engaging devices 400 may be delivered through one lumen before, after, or simultaneously with instruments delivered through another lumen of the medical delivery device 300. In embodiments of a traction system 200 delivered with a medical delivery device 300 having a single working channel, additional tissue-engaging devices 400 and other instruments may be delivered through the same working channel. For instance, the additional tissue-engaging devices 400 may be delivered to the treatment site T prior to delivery of a cutting instrument or other device (through the same single working channel, but at a different time).

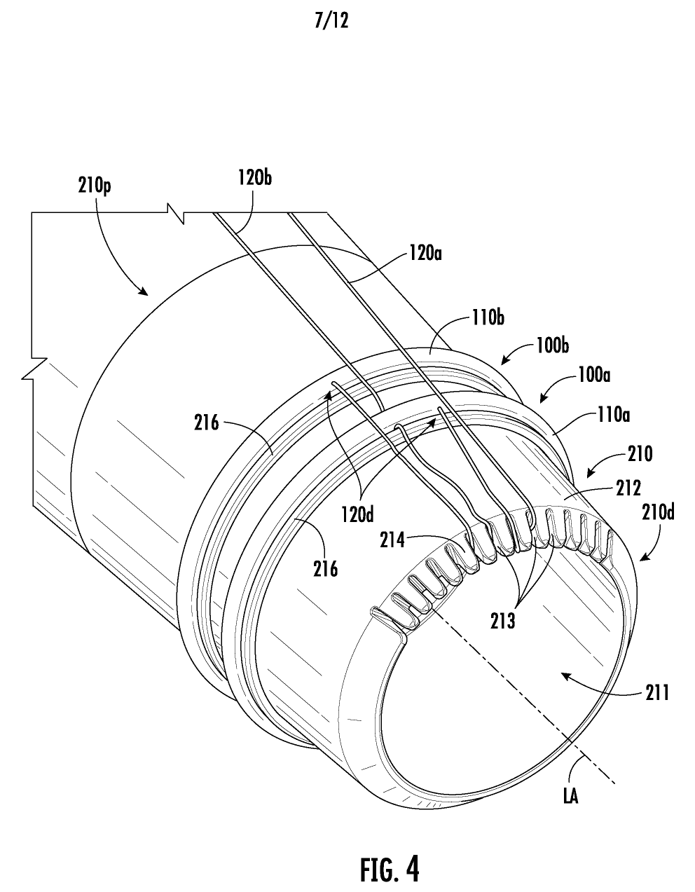

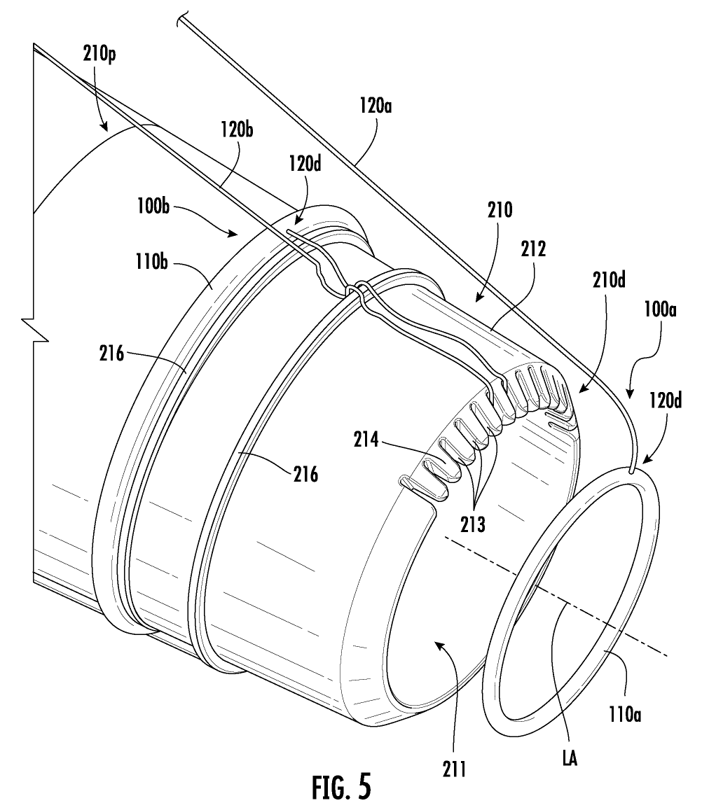

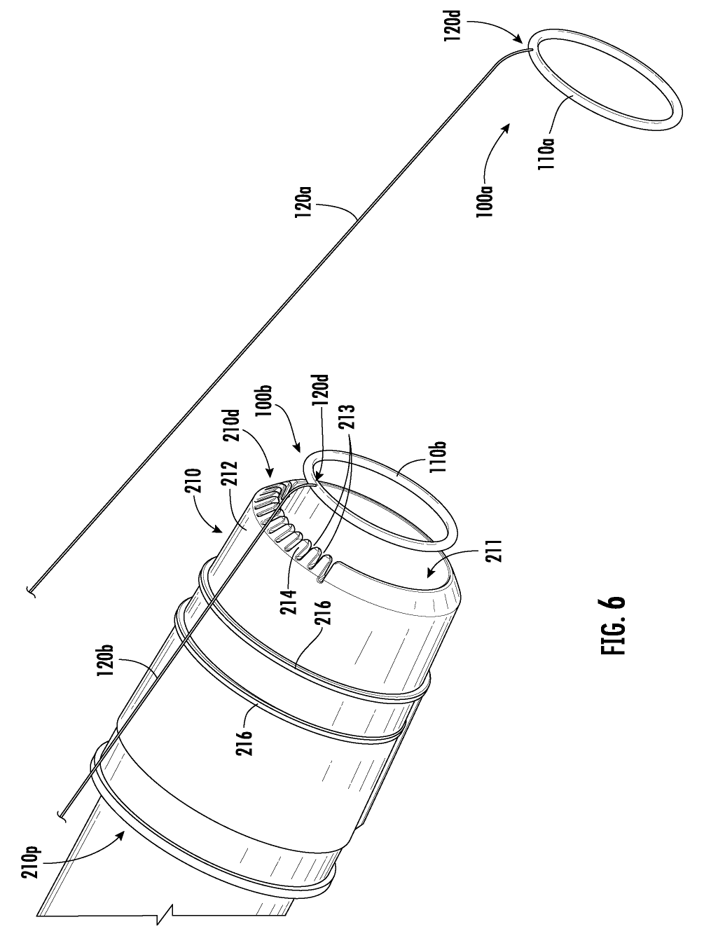

In the example of an embodiment illustrated in FIG. 1, the traction device 100 is delivered along the exterior of the medical delivery device 300. In some aspects, the anchor element 110 of the traction device 100 is delivered along the distal end 310d of the flexible tubular elongate member 310. In some aspects, the anchor element 110 of the traction device 100 is delivered extending around the external circumference of the distal end 310d of the flexible tubular elongate member 310. In the example of an embodiment of a traction device 100 illustrated in FIG. 1, a traction device 100 formed in accordance with various principles of the present disclosure may be delivered mounted on a distal interface member 210 of a traction system 200, such as illustrated in further detail in FIG. 4, FIG. 5, and FIG. 6. For example, the traction device 100 may be mounted on the exterior surface of the wall 212 of the distal interface member 210 of the traction system 200, such as extending circumferentially around at least a part of the circumference of the wall 212. As may be appreciated with reference to FIG. 1, the distal interface member 210 is configured to interface with the distal end 300d of the medical delivery device 300, such as with respect to the distal end 310d of the flexible tubular elongate member 310, to operably associate (e.g., couple or mount) the traction system 200 with respect to the medical delivery device 300. In the example of an embodiment illustrated in FIG. 1, FIG. 4, FIG. 5, and FIG. 6, the distal interface member 210 has an arcuate or tubular wall 212 extending at least partially around the exterior circumference of the distal end 310d of the flexible tubular elongate member 310. In some aspects, the distal interface member 210 is in the form of an endcap configured to be mounted over the distal end 310d of the flexible tubular elongate member 310 of the medical delivery device 300. In some aspects, the anchor element 110 of the traction device 100 is configured to be mounted with respect to the distal interface member 210 of the traction system 200. For instance, the anchor element 110 may be in the form of a band or ring or loop (e.g., any shape surrounding an open space or defining an open space therethrough) which may extend circumferentially with respect to the distal interface member 210 of the traction system 200, such as circumferentially around the exterior of the distal interface member 210 (e.g., with the distal interface member 210 extending through the open space through the anchor element 110).

The anchor element 110 of a traction device 100 formed in accordance with various principles of the present disclosure is deployed from the distal interface member 210 of the traction system 200 of the present disclosure by being moved distally off the distal end 210d of the distal interface member 210 in any of a variety of manners. Although a plurality of traction devices 100 may be mounted on the distal interface member 210 and deployed therefrom, the following description is with reference to a generic anchor element 110 and associated elongate element 120, without reference to a particular traction device 100a, 100b, etc. (traction devices 100 in addition to those which are illustrated). In the example of an embodiment illustrated in FIG. 4, FIG. 5, and FIG. 6, the elongate element 120 is manipulated to deploy the anchor element 110 from and off of the distal interface member 210. More particularly, the elongate element 120 is coupled with respect to the anchor element 110 and arranged to be pulled proximally to deploy the anchor element 110 distally. In some aspects, the anchor element 110 of a traction device 100 is deployed off the distal interface member 210 (e.g., off the exterior surface of the distal interface member 210) by proximal movement of its associated elongate element 120. In the example of an embodiment illustrated in FIG. 4, FIG. 5, and FIG. 6, the distal interface member 210 includes a direction-changing feature 214 around which the elongate element 120 extends. The elongate element 120 extends distally from its distal end 120d, coupled with the anchor element 110 at a location proximal to the distal end 210d of the distal interface member 210, to the direction-changing feature 214. In the example of an embodiment illustrated in FIG. 4, FIG. 5, and FIG. 6, the direction-changing feature 214 is at or along the distal end 210d of the distal interface member 210, although other locations, such as other locations distal to the anchor element 110, are within the scope of the present disclosure. The illustrated direction-changing feature 214 is formed between a pair of slits 213 formed along the distal end 210d of the distal interface member 210. The elongate element 120 extends distally and longitudinally along the exterior of the distal interface member 210, through a slit 213 on one side of a direction-changing feature 214, laterally (orthogonal to the longitudinal axis LA of the distal interface member 210) to the slit 213 on the other side of the direction-changing feature 214, then proximally and longitudinally along the exterior of the distal interface member 210¸ and proximally beyond the proximal end 210p of the distal interface member 210 and proximally along the medical delivery device 300 to outside the patient’s body where a medical professional may manipulate the elongate element 120 (e.g., grasp a proximal end of the elongate element 120 or manipulate an element to which the elongate element 120 is operably coupled, as described in further detail below). With such arrangement of the anchor element 110, the elongate element 120, and the distal interface member 210, the medical professional may move the elongate element 120 proximally to effect distal movement of the anchor element 110 to deploy the anchor element 110 off the distal interface member 210.

As may be appreciated with reference to FIG. 4, more than one traction device 100a, 100b may be delivered on, by, with, etc., the distal interface member 210. A plurality of slits 113 are formed along the distal end 210d of the distal interface member 210 to form a plurality of direction-changing features 214, preferably to correspond with each elongate element 120. Each traction device 100 which is delivered over the distal interface member 210 may be delivered in a similar manner as described above. The traction devices 100 typically are delivered sequentially, with a distalmost traction device 100 being delivered before the next proximal traction device 100. So that the elongate elements 120 of the proximal traction devices 100 do not interfere with operation of the elongate element 120 of the distalmost anchor element 110 to deploy the distalmost anchor element 110, the distalmost anchor element 110 is positioned over the elongate elements 120 of the more proximal anchor elements 110. Each anchor element 110 of a plurality of anchor elements 110 mounted on the distal interface member 210 may be positioned over the elongate elements 120 of more proximal traction devices 100. For instance, as illustrated in FIG. 4, the anchor element 110a of the distal traction device 100 is positioned over the elongate element 120b of the proximal traction device 100b.

The elongate element 120a of the distal traction device 100a may thus be operated without interference by the anchor element 110b or the elongate element 120b of the proximal traction device 100b.

Once the distal traction device 100a has been deployed, as illustrated in FIG. 5, the elongate element 120b of the proximal traction device 100b is free to be manipulated to deploy the associated proximal anchor element 110b, such as illustrated in FIG. 6. Deployment of the distal traction device 100 may be in a manner as described above, reference being made to the above description for the sake of brevity and without intent to limit. The deployed traction devices 100 may be anchored with respect to target tissue F at a treatment site T such as illustrated in FIG. 3 (which illustrates a view through the lumen 211 of the distal interface member 210, and which shows another perspective of the direction-changing features 214 and slits 213 through the wall 212 of the distal interface member 210).

In some aspects, the exterior surface of the distal interface member 210 is distally tapered to facilitate deployment of the traction devices 100. In some aspects, the exterior surface of the distal interface member 210 is provided with a retaining feature such as a stopper 216 extending radially outwardly from the distal interface member 210 and distal to the anchor element 110 to inhibit unintended distal movement of the anchor element 110 off the distal interface member 210. With such stopper 216, the medical professional must apply sufficient force to pull the anchor element 110 over the stopper 216 (e.g., to radially expand over and longitudinally across the stopper 216). The stopper 216 may be in the form of a circumferentially extending rib or ridge which projects radially outwardly from the exterior surface of the distal interface member 210, or may be one or more projections which do not extend around the full circumference of the distal interface member 210, or any other configuration which retains the anchor element 110 in place with respect to the distal interface member 210 until sufficient intended force is applied to dislodge and displace the anchor element 110 distally with respect to the stopper 216 to be deployed off the distal interface member 210. A stopper 216 may be provided for each anchor element 110 mounted with respect to the distal interface member 210.

As illustrated in FIG. 5 and FIG. 6, the anchor element 110 may be configured to maintain a shape which allows a grasper instrument to grasp the anchor element 110. For instance, the anchor element 110 may be formed of a self-supporting material. In the example of an embodiment illustrated in FIG. 5, the anchor element 110 is formed in a ring or loop shape encircling an opening / aperture, and is sufficiently self-supporting to maintain such ring or loop shape so that a grasper instrument (e.g., an element with a hook or jaws) can be extended through the opening / aperture to grasp the anchor element 110. For instance, one jaw 402 of a tissue-engaging device 400 such as described above may be extended through the opening / aperture of the anchor element 110 and the other jaw may be positioned on a radially-outward side of the anchor element 110. The jaws 402 (optionally partially or fully closed to grasp the anchor element 110) may then move the anchor element 110 to the target tissue (to which traction is to be applied). The jaws 402 may then be manipulated to engage the target tissue, thereby coupling the anchor element 110 to the target tissue as well, such as described above with reference to FIGS. 2A-2D. In some aspects, a grasper with a releasable tissue-engaging device 400 may be used so that the end effector of the grasper is also the tissue-engaging device 400 which is deployed with the anchor element 110 at the target tissue. Examples of such tissue-engaging devices include, but are not limited to, those described in U.S. Patent 7,494,461, issued February 24, 2009, and titled “Through The Scope Tension Member Release Clip”; U.S. Patent 8,062,311, issued November 22, 2011, and titled “Endoscopic Hemostatic Clipping Apparatus”; U.S. Patent 8,080,021, issued December 20, 2011, and titled “Multiple Clip Deployment Magazine”; and U.S. Patent Application Publication 2009/0187198, filed December 15, 2008, and titled “Resolution Clip”, all of which are herein incorporated by reference in their entirety and for all purposes. Once the tissue-engaging device 400 is engaged with target tissue and anchors the anchor element 110 with respect to the target tissue as well, the tissue-engaging device 400 is released from a control shaft used to manipulate and control the tissue-engaging device 400 of the grasper, and the tissue-engaging device 400 with the anchor element 110 are deployed at the target tissue.

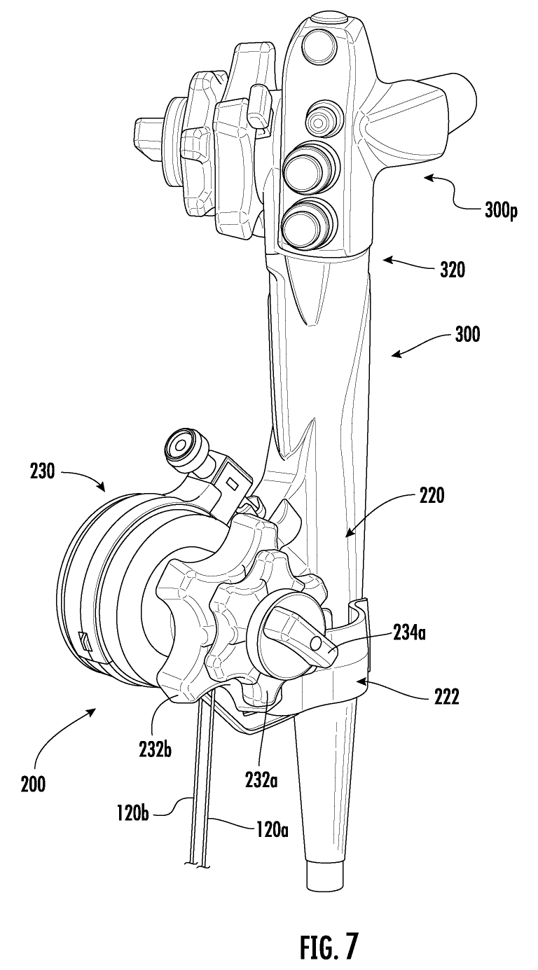

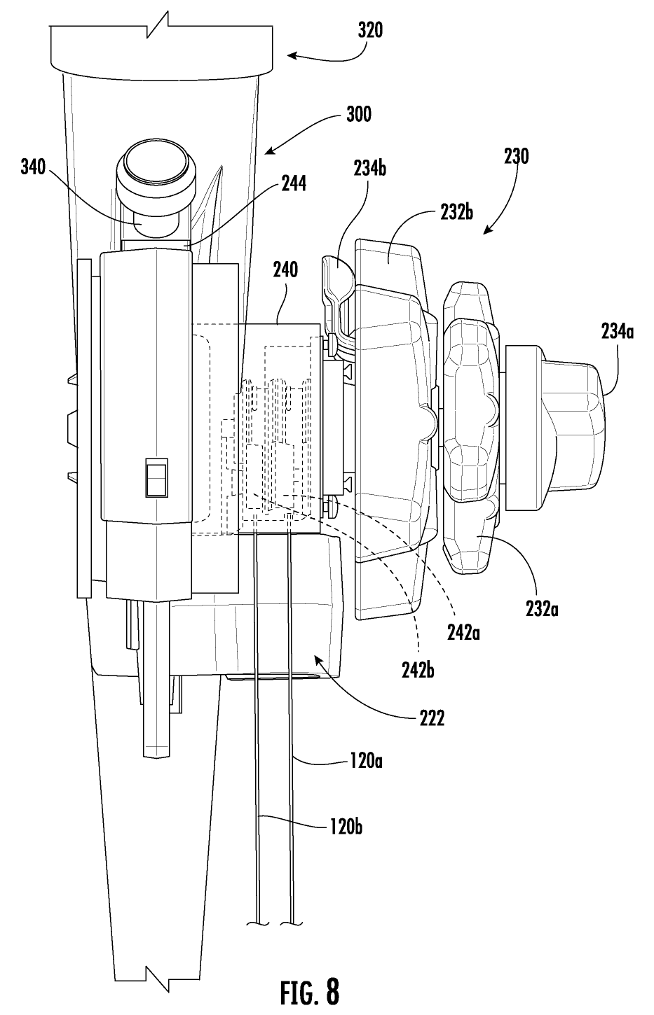

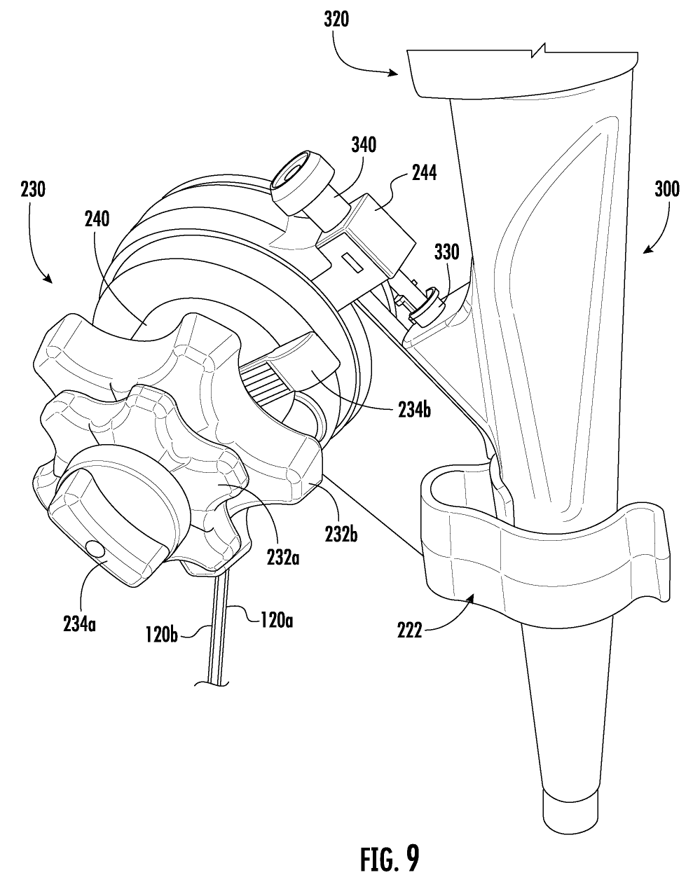

In some aspects, a medical professional may (e.g., manually) grasp a proximal end of the elongate element 120 of a traction device 100 to deploy and/or apply traction to the anchor element 110 of the traction device 100. In some aspects, a traction system 200 formed in accordance with various principles of the present disclosure includes a proximal interface member 220 configured to operably associate one or more traction devices 100 of the present disclosure with the medical delivery device 300, such as illustrated in FIG. 1, FIG. 7¸ FIG. 8, and FIG. 9. For instance, the proximal interface member 220 may be mounted with respect to the medical delivery device 300 with a mount such as a c-clip or bracket 222, and include a control section 230 operably associated with the elongate elements 120 the of one or more traction devices 100 to be used. The proximal interface member 220 may thus maintain the proximal end of the elongate elements 120 of the traction devices 100 along or at a location near the control handle 320 of the medical delivery device 300. Such arrangement facilitates access and manipulation of the traction device 100 by the medical professional operating the medical delivery device 300. Thus, an assistant / second medical professional does not need to operate / manipulate the traction device 100 while a first medical professional operates / manipulates the medical delivery device 300 and associated medical instruments. Instead, the same medical professional can more readily operate / manipulate all devices, including the medical delivery device 300, the traction device 100, and additional medical instruments delivered through the lumen of the medical delivery device 300.

In some aspects, the proximal interface member 220 includes one or more control elements 232, such as control knobs 232, which may assist the medical professional in operating, manipulating, controlling, etc., the traction device 100. It will be appreciated that terms such as operate, manipulate, control, etc., including other grammatical forms thereof, may be usable interchangeably herein without intent to limit unless specified. In some aspects, the elongate element 120 of a traction device 100 is operably coupled with a control knob 232 such that the control knob 232 may be operated by the medical professional to apply and/or control a force applied to the elongate element 120, such as to apply traction to the anchor element 110 and target tissue F to which the anchor element 110 is anchored (such as schematically illustrated in FIG. 2D). For instance, each traction device 100a, 100b, etc., may have an associated control knob 232a, 232b, etc. In some aspects, more than one control knob 232 is provided so that more than one traction device 100 may be delivered by the traction system 200, as well as controlled individually and separately by a respective control knob 232 of the traction system 200. In some aspects, the traction device 100 to be deployed first (e.g., the distal traction device 100a in the example of an embodiment illustrated in FIGS. 4-6) may be associated with the proximal control knob 232a to be accessed first by the medical professional. The next control knob 232b (adjacent but distal to the proximal control knob 232a) may be associated with the next traction device 100b to be deployed. As may be appreciated, a control knob 232 may be provided for each traction device 100, with the control knobs 232 optionally arranged in a sequential order, such as to be operated in sequence to deploy a traction device 100 of a plurality of traction devices 100 in a sequence. It will be appreciated that although the arrangement of control knobs 232 is illustrated as proximal to distal to correspond with traction devices 100 deployed from distalmost to proximalmost traction device 100, the present disclosure is not limited in this regard.

The elongate elements 120 of the traction devices 100 controlled by the control knobs 232 may be operably associated with the control knobs 232 in any of a variety of manners. In the example of an embodiment illustrated in FIG. 8, the elongate elements 120a, 120b are respectively operably associated with pulleys 242a, 242b which are operably associated with the respective control knobs 232a, 232b. The pulleys 242a, 242b may be axles, spools, etc., about which the elongate elements 120a, 120b respectively extend (e.g., are wrapped circumferentially around), and which are operated (e.g., turned), respectively, by the control knobs 232a, 232b to modify the length of the elongate elements 120a, 120b between the control knobs 232a, 232b and the anchor elements 110a, 110b, respectively. The pulleys 242a, 242b may be rotated by their associated control knobs 232a, 232b to proximally pull and reel in an elongate element 120, such as to apply traction to the associated anchor element 110 and tissue to which the anchor element 110 is anchored, or to release and unreel or pay out a portion of the elongate element 120 which had been wound about a pulley 242a, 242b, such as to release traction on the associated anchor element 110 and tissue to which the anchor element 110 is anchored. The pulleys 242a, 242b may be housed within a housing 240 associated with the control section 230 of the proximal interface member 220. As may be appreciated with reference to FIG. 9, the housing 240 may include a bracket 244 configured to mount the housing 240, and thus the proximal interface member 220, with respect to the proximal end 300p of the medical delivery device 300, such as with respect to the control handle 320 of the medical delivery device 300. In the example of an embodiment illustrated in FIG. 9, the bracket 244 is mounted with respect to the port 330 of the medical delivery device 300. In some aspects, a port extension 340 (e.g., a tubular member such as a cannula) is operably coupled with the port 330 (e.g., a distal portion of the port extension 340 may be inserted and secured within the port 330) to facilitate mounting of the bracket 244 with respect to the port 330 via the port extension 340.

Once the desired amount of force has been applied to a traction device 100 of the present disclosure, such as through the use of an associated control knob 232, to achieve the desired amount of traction on the target tissue, a lock 234 may be actuated to fix the position of the control knob 232. Each control knob 232a, 232b may have an associated lock 234a, 234b, which may be positioned adjacent its associated control knob 232a, 232b. In the example of an embodiment illustrated in FIG. 1, FIG. 7, FIG. 8, and FIG. 9, the lock 234a operably associated with the control knob 232a for the first traction device 100a to be deployed (in the example of an embodiment illustrated in FIGS. 4-6, the distal traction device 100a) is positioned proximal to the control knob 232a. In the example of an embodiment illustrated in FIGS. 7-9, the lock 234b operably associated with the control knob 232b for the proximal traction device 100b may be adjacent (e.g., proximal) to the control knob 232b. As may be more clearly seen in FIG. 9, the lock 234b for the proximal traction device 100b may extend radially-outwardly with respect to the control knob 232b. It will, however, be appreciated that the present disclosure need not be limited to the arrangements and/or relative positions of the control knobs 232 and/or associated locks 234a, 234b. It will be appreciated that the locks 234a, 234b may hold an associated one of the control knobs 232a, 232b in any of a variety of manners, such as a friction lock, a wedge mechanism, interlocking features (e.g., square teeth), or other type of locking mechanism known to those of ordinary skill in the art, the present disclosure not being limited in this regard. For instance, the locks 234a, 234b may operate similar to locks of the control handle 320 of a medical delivery device 300 such as an endoscope. In some aspects, the locks 234a, 234b are biased into a locking configuration, holding the control knobs 232a, 232b, respectively, against movement.

As may be appreciated in view of the above, the traction device 100 and/or the traction system 200 of the present disclosure is configured to decouple the operation of the traction device 100 from the medical delivery device 300 and its operation. In some aspects, delivery, deployment, and operation of the traction device 100 are independent of delivery, deployment, and operation of other medical instruments delivered by the medical delivery device 300. For instance, in some aspects, a traction system 200 of the present disclosure is used in conjunction with a medical delivery device 300 which has a flexible tubular elongate member 310 defining a lumen (e.g., working channel) therethrough. The lumen of the flexible tubular elongate member 310 may be configured for passage of one or more medical instruments therethrough for use at a treatment site at which the distal end 300d of the medical delivery device 300 is positioned. In some aspects, the traction device 100 is deliverable independently of the lumen though the flexible tubular elongate member 310 of the medical delivery device 300, such as via a path other than through the lumen of the flexible tubular elongate member 310. In some aspects, the traction device 100 is delivered outside the lumen of the medical delivery device 300, such as along the exterior of the medical delivery device 300. As such, the traction device 100 is deliverable independently of delivery of medical devices through the lumen of the medical delivery device 300. Moreover, the traction device 100 is operable independently of operation of medical instruments which are advanced distally through the lumen of the flexible tubular elongate member 310 for use at the distal end 300d of the medical delivery device 300.

Additionally or alternatively, as may be appreciated in view of the above, a traction system 200 and/or a traction device 100 of the present disclosure is configured so that multi-vector traction, such as with the use of two or more traction device 100, may be applied to tissue at a treatment site. In some aspects, the multi-vector traction is applied independently of the operation of one or more medical instruments operated at the treatment site. For instance, the multi-vector traction may be applied to the traction devices 100 independently of operation of one or more medical instruments extended through a flexible tubular elongate member 310 of a medical delivery device 300 with which the traction devices 100 are delivered. In some aspects, the elongate element 120 of the traction device 100 is extendable outside the flexible tubular elongate member 310 to a proximal end outside the patient and accessible for control by a medical professional (e.g., manipulation and/or application of force thereto).

Although the present disclosure describes medical devices and systems and procedures for performing an ESD procedure, it should be appreciated that medical devices, systems, and methods of the present disclosure may be used to treat various anatomical tissues in any of a variety of medical procedures.

It is to be understood by one of ordinary skill in the art that the above descriptions are of examples of embodiments only, and are not intended as limiting the broader aspects of the present disclosure. The devices, systems, and methods discussed herein are not the only way to implement the various principles of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims. Various further benefits of the various aspects, features, components, and structures of traction devices, systems, and methods such as described above, in addition to those discussed above, may be appreciated by those of ordinary skill in the art.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and / or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description. Tubular elongate member 1200 tubular elongate member 1200

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader’s understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A traction system for delivery into a patient’s body with a medical delivery device having a lumen for delivering medical instruments through a distal end of the medical delivery device, said traction system comprising:
a distal interface member comprising a wall configured to interface with the medical delivery device;
a first anchor element mounted on an exterior surface of said distal interface member; and
a first elongate element operably associated with said first anchor element and extendable proximally from said distal interface member for access outside the patient’s body.

2. The traction system of claim 1, wherein said first anchor element extends circumferentially around the exterior surface of said distal interface member.

3. The traction system of claim 2, wherein said first anchor element is in the form of a band or loop encircling an opening.

4. The traction system of claim 3, wherein said first anchor element is structurally self-supporting to maintain the opening in an open configuration without the first anchor folding on itself.

5. The traction system of claim 1, wherein said first anchor element is structurally self-supporting to maintain a selected shape without folding on itself.

6. The traction system of claim 1, wherein said distal interface member is configured to extend circumferentially around the exterior of a portion of the medical delivery device.

7. The traction system of claim 6, wherein said wall of said distal interface member is tubular and shaped to extend circumferentially around the distal end of the medical delivery device.

8. The traction system of claim 1, wherein a first radially-outwardly extending stopper is defined on the exterior surface of said distal interface member to retain said first anchor element in place with respect to said distal interface member.

9. The traction system of claim 8, wherein said first radially-outwardly extending stopper is an elevated ridge extending circumferentially around the exterior of said distal interface member.

10. The traction system of claim 1, wherein said first elongate element extends distally from said first anchor element, and around a direction-changing feature defined with respect to said distal interface member to change directions to extend proximally to outside the patient’s body.

11. The traction system of claim 10, wherein said direction-changing feature is defined between a pair of slits in a distal end of said distal interface member.

12. The traction system of claim 1, further comprising a second anchor element mounted on the exterior surface of said distal interface member, and a second elongate element operably associated with said second anchor element and extendable proximally from said anchor element on said distal interface member for access outside the patient’s body.

13. A traction system for delivery into a patient’s body, said traction system comprising:
a proximal interface member configured to interface with a medical delivery device;
a distal interface member comprising a wall configured to interface with the medical delivery device;
a first anchor element mounted on an exterior surface of said distal interface member; and
a first elongate element operably associated with said first anchor element and extendable proximally from said distal interface member to said proximal interface member.

14. The traction system of claim 13, wherein said first anchor element and said first elongate element are preassembled on said distal interface member such that said traction system is assembled and ready for operable association with a medical delivery device to operably associate said first anchor element and said first elongate element with the medical delivery device.

15. The traction system of claim 13, further including a medical delivery device having a lumen for delivering medical instruments through a distal end of the medical delivery device, wherein said first elongate element extends along the exterior of said medical delivery device outside the lumen of the medical delivery device.

16. The traction system of claim 13, further comprising a second anchor element mounted on the exterior surface of said distal interface member, and a second elongate element operably associated with said second anchor element and extendable proximally from said anchor element on said distal interface member for access outside the patient’s body.

17. A method of assembling a traction device with respect to a medical delivery device having a lumen for delivering medical instruments through a distal end of the medical delivery device, said method comprising:
mounting a distal interface member with respect to the distal end of the medical delivery device, the distal interface member having a first anchor element of a traction device extending circumferentially around the exterior of the distal interface member; and
extending a first elongate element from a distal end operably coupled with the first anchor element to the proximal end of the medical delivery device.

18. The method of claim 17, wherein the distal interface member includes a direction-changing feature, the first elongate element extending distally from the first anchor element to around the direction-changing feature, and then proximally.

19. The method of claim 17, wherein the distal interface member includes a second anchor element positioned proximal to the first anchor element, with a second elongate element operably associated with the second anchor element and extending proximally.

20. The method of claim 17, further comprising mounting a proximal interface member with respect to a proximal end of the medical delivery device and extending the first elongate element to the proximal interface member for control of the elongate element along the proximal interface member.
